(19) United States
(12) Patent Application Publication
(10) Pub. No.: US 2024/0245331 A1
(43) Pub. Date: Jul. 25, 2024

(54) ANALYTE MONITORING DEVICE AND METHODS

(71) Applicant: ABBOTT DIABETES CARE INC., Alameda, CA (US)

(72) Inventors: Jean-Pierre Cole, Tracy, CA (US); Martin J. Fennell, Concord, CA (US)

(21) Appl. No.: 18/374,245

(22) Filed: Sep. 28, 2023

Related U.S. Application Data
(63) Continuation of application No. 15/961,448, filed on Apr. 24, 2018, now abandoned.
Application No. 15/961,448 is a continuation of application No. 13/671,489, filed on Nov. 7, 2012, now Pat. No. 9,980,669.
(60) Provisional application No. 61/556,824, filed on Nov. 7, 2011.

(51) Int. Cl. A61B 5/1473; A61B 5/00; A61B 5/145; A61B 5/1486

(52) U.S. Cl. CPC A61B 5/1473; A61B 5/0026; A61B 5/14532; A61B 5/14865; A61B 5/72; A61B 5/7225; A61B 5/0004; A61B 5/6833

(57) ABSTRACT

Methods and devices for providing application specific integrated circuit architecture for a two electrode analyte sensor or a three electrode analyte sensor are provided. Systems and kits employing the same are also provided.

### CROSS-REFERENCE TO RELATED APPLICATIONS

This application is a continuation of U.S. patent application Ser. No. 15/961,448, filed Apr. 24, 2018, which is a continuation of U.S. patent application Ser. No. 13/671,489, filed Nov. 7, 2012, now U.S. Pat. No. 9,980,669, which claims priority to, and the benefit of, U.S. Provisional Patent Application 61/556,824 filed Nov. 7, 2011, the contents of all of which are hereby incorporated by reference herein in their entireties and for all purposes.

### BACKGROUND

The detection and/or monitoring of glucose levels or other analytes, such as lactate, oxygen, A1C, or the like, in certain individuals is vitally important to their health. For example, the monitoring of glucose is particularly important to individuals with diabetes. Diabetics generally monitor glucose levels to determine if their glucose levels are being maintained within a clinically safe range, and may also use this information to determine if and/or when insulin is needed to reduce glucose levels in their bodies or when additional glucose is needed to raise the level of glucose in their bodies.

Growing clinical data demonstrates a strong correlation between the frequency of glucose monitoring and glycemic control. Despite such correlation, many individuals diagnosed with a diabetic condition do not monitor their glucose levels as frequently as they should due to a combination of factors including convenience, testing discretion, pain associated with glucose testing, and/or cost.

Devices have been developed for the automatic or continuous monitoring of analyte(s), such as glucose, in bodily fluid such as in the blood stream or in interstitial fluid (“ISF”), or other biological fluid. Some of these analyte measuring devices are configured so that at least a portion of the devices are positioned below a skin surface of a user, e.g., in a blood vessel or in the subcutaneous tissue of a user, so that the monitoring is accomplished in vivo.

With the continued development of analyte monitoring devices and systems, there is a need for such analyte monitoring devices, systems, and methods, as well as for processes for manufacturing analyte monitoring devices and systems that are cost effective, convenient, and with reduced pain, provide discreet monitoring to encourage frequent analyte monitoring to improve glycemic control.

### SUMMARY

In view of the foregoing, devices, methods and systems for providing electronics for coupling to analyte sensors are provided including, for example, application specific integrated circuit (ASIC) configurations that provide electrical coupling with electrochemical sensors such as, for example, in vivo glucose sensors for continuous monitoring of analytes such as glucose.

These and other objects, features and advantages of the present disclosure will become more fully apparent from the following detailed description of the embodiments, the appended claims and the accompanying drawings.

### BRIEF DESCRIPTION OF THE DRAWINGS

FIG. 1 shows an overall in vivo sensor based analyte monitoring system for use in certain embodiments of the present disclosure;

FIG. 2 is an illustration of the analog front end architecture for electrical coupling to analyte sensor electrodes in an analyte monitoring system in certain embodiments of the present disclosure;

FIG. 3 is another illustration of the analog front end architecture for electrical coupling to analyte sensor electrodes in an analyte monitoring system in certain embodiments of the present disclosure;

FIG. 4 is yet another illustration of the analog front end architecture for electrical coupling to analyte sensor electrodes in an analyte monitoring system in certain embodiments of the present disclosure;

FIG. 5 is yet still another illustration of the analog front end architecture for electrical coupling to analyte sensor electrodes in an analyte monitoring system in certain embodiments of the present disclosure;

FIG. 6 is yet still a further illustration of the analog front end architecture for electrical coupling to analyte sensor electrodes in an analyte monitoring system in certain embodiments of the present disclosure; and

FIG. 7 is yet still another illustration of the analog front end architecture for electrical coupling to analyte sensor electrodes in an analyte monitoring system in certain embodiments of the present disclosure.

### DETAILED DESCRIPTION

Before the present disclosure is described in detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

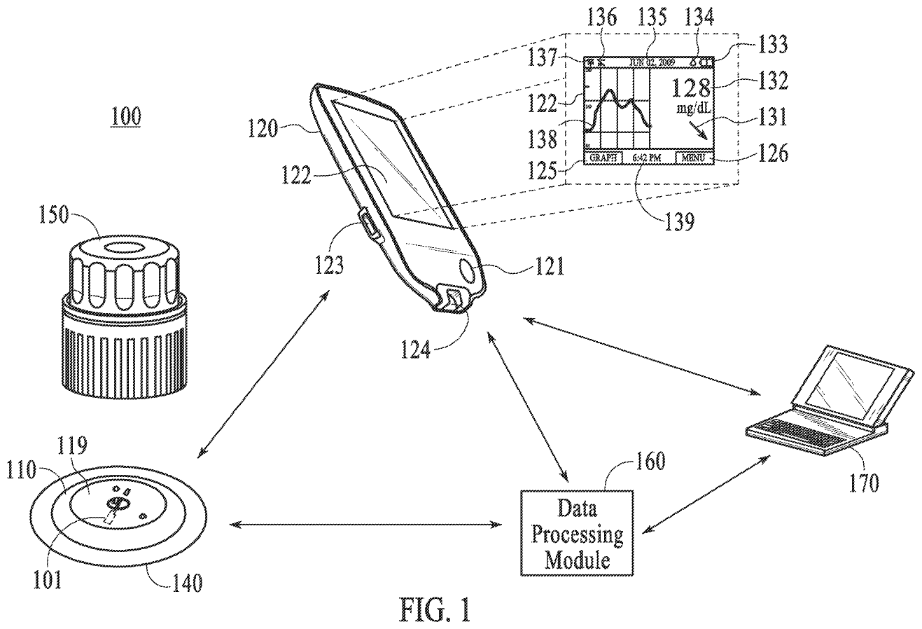

FIG. 1 shows an exemplary in vivo based analyte monitoring system 100 in accordance with embodiments of the present disclosure. As shown, in certain embodiments, analyte monitoring system 100 includes on body electronics 110 electrically coupled to in vivo analyte sensor 101 (a proximal portion of which is shown in FIG. 1) and attached to adhesive layer 140 for attachment on a skin surface on the body of a user. On body electronics 110 includes on body housing 119, that defines an interior compartment. Also shown in FIG. 1 is insertion device 150 that, when operated, transcutaneously positions a portion of analyte sensor 101 through a skin surface and in fluid contact with ISF, and positions on body electronics 110 and adhesive layer 140 on a skin surface. In certain embodiments, on body electronics 110, analyte sensor 101, and adhesive layer 140 are sealed within the housing of insertion device 150 before use, and in certain embodiments, adhesive layer 140 is also sealed within the housing or itself provides a terminal seal of the insertion device 150. Devices, systems and methods that maybe used with embodiments herein are described, e.g., in U.S. patent application Ser. Nos. 12/698,124, 12/698,129 and 12/807,278, the disclosures of each of which are incorporated herein by reference for all purposes.

Referring back to the FIG. 1, analyte monitoring system 100 includes display device 120 which includes a display 122 to output information to the user, an input component 121 such as a button, an actuator, a touch sensitive switch, a capacitive switch, a pressure sensitive switch, a jog wheel or the like, to input data or commands to display device 120, or otherwise control the operation of display device 120.

In certain embodiments, input component 121 of display device 120 may include a microphone and display device 120 may include software configured to analyze audio input received from the microphone, such that functions and operation of the display device 120 may be controlled by voice commands. Display device 120 also includes data communication port 123 for wired data communication with external devices such as remote terminal (personal computer) 170, for example. Display device 120 may also include an integrated in vitro glucose meter, including in vitro test strip port 124 to receive an in vitro glucose test strip for performing in vitro blood glucose measurements.

Referring still to FIG. 1, display 122 in certain embodiments is configured to display a variety of information—some or all of which may be displayed at the same or different time on display 122. Display 122 may include, but is not limited to, graphical display 138, numerical display 132, trend or directional arrow display 131, date display 135, time of day information display 139, battery level indicator display 133, sensor calibration status icon display 134, mute on/off icon display 136, and wireless connectivity status icon display 137 that provides indication of wireless communication connection with other devices such as on body electronics 110, data processing module 160, and/or remote terminal 170. As additionally shown in FIG. 1, display 122 may further include simulated touch screen button 125, 126 for accessing menus, changing display graph output configurations or otherwise for controlling the operation of display device 120.

Further details and other display embodiments can be found in, e.g., U.S. patent application Ser. Nos. 12/871,901 and 12/807,278, the disclosures of each of which are incorporated herein by reference for all purposes.

After the positioning of on body electronics 110 on the skin surface and analyte sensor 101 in vivo to establish fluid contact with ISF (or other appropriate body fluid), on body electronics 110 in certain embodiments is configured to wirelessly communicate analyte related data (such as, for example, data corresponding to monitored analyte level and/or monitored temperature data, and/or stored historical analyte related data) when on body electronics 110 receives a command or request signal from display device 120. In certain embodiments, data from on body electronics 110 is retrieved using display device 120 or a reader device via a wireless link that operates using a near field reflective communication technique, such as is used in radio frequency identification (RFID) systems. Using such systems, in certain embodiments, analyte measurement from analyte sensor 101 can be obtained by positioning display device 120 within a short range of on body electronics 110, and optionally actuating a button such as input component 121 to initiate data transfer from on body electronics 110 to display device 120.

In certain embodiments, the RFID communication operates at a nominal operating frequency of 13.56 MHz, with minimum antenna input voltage for normal operation at about 2.5 Volts. Data rate for transmit and receive operations between on body electronics 110 and display device 120 may be about 20-30 kbits/second, or about 22-28 kbits/second, or about 26.48 kbits/second (data bits) in certain embodiments.

In certain embodiments, on body electronics 110 may be configured to at least periodically broadcast real time data associated with monitored analyte level which is received by display device 120 when display device 120 is within communication range of the data broadcast from on body electronics 110, i.e., on body electronics 110 does not need a command or request from display device 120 to send information.

In certain embodiments, the received data from on body electronics 110 may be stored (permanently or temporarily) in one or more memory of the display device 120. Referring still to FIG. 1, also shown in analyte monitoring system 100 are data processing module 160 and remote terminal 170. Remote terminal 170 may include a personal computer, a server terminal, a laptop computer, or other suitable data processing devices including software for data management and analysis and communication with the components in analyte monitoring system 100.

Data processing module 160 may include components to communicate using one or more wireless communication protocols such as, for example, but not limited to, infrared (IR) protocol, Bluetooth® protocol, Zigbee® protocol, and 802.11 wireless LAN protocol. Additional description of communication protocols including those based on Bluetooth® protocol and/or Zigbee® protocol can be found in U.S. Patent Publication No. 2006/0193375 incorporated herein by reference for all purposes.

In a further aspect, software algorithms for execution by data processing module 160 may be provided to a communication device such as a mobile telephone including, for example, WiFi or Internet enabled smart phones or personal digital assistants (PDAs) as a downloadable application for execution by the downloading communication device. Additional details describing field upgradability of software of portable electronic devices, and data processing are provided in U.S. patent application Ser. Nos. 12/698,124, 12/794,721, 12/699,653, and 12/699,844, and U.S. Provisional Application Nos. 61/359,265 and 61/325,155, the disclosures of each of which are incorporated by reference herein for all purposes.

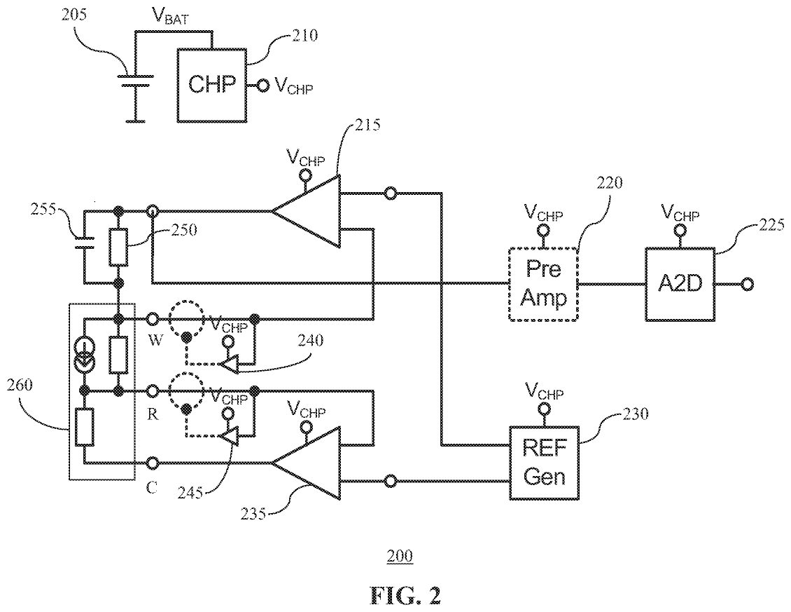

FIG. 2 is an illustration of the analog front end architecture for electrical coupling to analyte sensor electrodes in an analyte monitoring system in certain embodiments of the present disclosure. Referring to FIG. 2, shown is analyte sensor 260 including working electrode W, reference electrode R and counter electrode C, each of which are operatively coupled to the analog front end circuitry 200. More specifically, referring to FIG. 2, working electrode W of analyte sensor 260 is electrically coupled to the input terminal of transimpedance amplifier 215, the reference electrode R is electrically coupled to the input terminal of the servo amplifier 235, and counter electrode C is electrically coupled to the output terminal of the servo amplifier 235.

Referring back to FIG. 2, analog front end circuitry 200 is powered with a battery 205 (Vbat) that is coupled to a charge pump 210 which is configured to boost the voltage received from the battery 205 and provide the boosted voltage VChp to the other components of the analog front end circuitry 200. For example, in certain embodiments, the battery 205 is a 1.5 Volt battery, which, when coupled to the charge pump 210, is boosted to 3V (as output from the charge pump 210), and thereafter, provided to the transimpedance amplifier 215, the servo amplifier 235, pre-amplifier 220, the analog-to-digital converter 225, the reference generator 230, as well as amplifiers 240, 245 that are configured with very high input impedance (e.g., 100 GigaOhms), to provide guard trace around the analog front end circuit connections as discussed in further detail below.

Referring again to FIG. 2, transimpedance amplifier 215 in certain embodiments, provides a fixed potential/voltage on the working electrode, for example, but not limited to approximately 2 volts+/−50 micro Volts, and converts the current signal from the output of transimpedance amplifier 215 into a measurable voltage through resistor 250 that is part of the transimpedance amplifier 215. Also shown in FIG. 2 is a capacitor 255 coupled in parallel to resistor 250 for the voltage signal at the output of the transimpedance amplifier 215. The voltage signal at the output of the transimpedance amplifier 215 is provided to the optional pre-amplifier 220 as shown in FIG. 2, where the pre-amplifier 220 in certain embodiments, buffers the received voltage signal and then provides the buffered voltage signal to the analog-to-digital converter 225, which thereafter outputs the converted signal for further processing by the on body electronics 110 (FIG. 1). In certain embodiments, the pre-amplifier 220 has high impedance (e.g., 1 GigaOhms) that will compensate for any signal variation in the output of the transimpedance amplifier 215 which is input to the pre-amplifier 220.

Referring still to FIG. 2, in certain embodiments, reference generator 230 is coupled to transimpedance amplifier 215 and servo amplifier 235, where the reference generator 230 provides the two input voltages (2 Volts+/−50 micro Volts, and 1.96 Volts+/−20 micro Volts, respectively) to the transimpedance amplifier 215 and servo amplifier 235. Accordingly, a 40 mVolt differential is maintained between the working electrode W and the reference electrode R of the analyte sensor 260. More specifically, the servo amplifier 235 in certain embodiments is controlled to 1.96 Volts+/−20 micro Volts, which is 40 mVolts below the 2 Volt (at which the transimpedance amplifier 215 is maintained), where the difference of 40 mVolts is the poise voltage for the analyte sensor 260 that is maintained between the working electrode W and the reference electrode R. In certain embodiments, as the analyte sensor current fluctuates, the servo amplifier 235 varies its output so that it maintains the 40 mVolts differential between the working electrode W and the reference electrode R of the analyte sensor 260.

Referring still again to FIG. 2, also shown are amplifiers 240 and 245, each receiving the boosted voltage Vchp from the charge pump 210, and, in certain embodiments, is configured as a unity gain amplifier or a voltage follower, that is very accurate, such that they maintain zero voltage difference between the electrode connection (for example, between the reference/working electrodes and the guard trace (represented by broken circular ring around the working and reference electrode connections). More specifically, the guard traces as shown in FIG. 2 in certain embodiments are provided to surround the entire circuit connections on the high impedance portion of the analog front end circuitry of the on body electronics 110. Without the guard traces as shown in FIG. 2 and described above, if there is any leakage to the circuit connections, measurement error will result, and further, in the case of leakage to the reference electrode, in certain embodiments, the silver/silver-chloride (Ag/AgCl) will degrade, effectively degrading the performance of the analyte sensor 260.

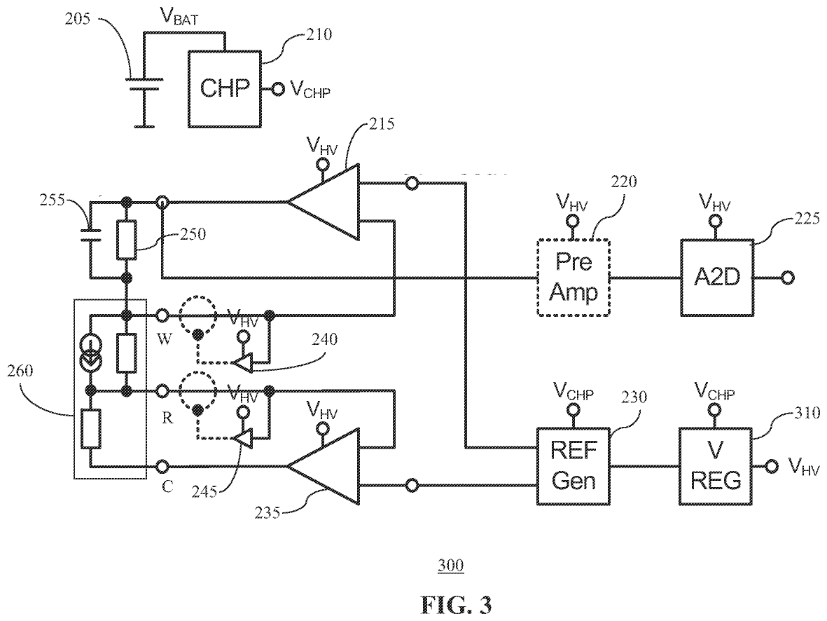

FIG. 3 is another illustration of the analog front end architecture 300 for electrical coupling to analyte sensor electrodes in an analyte monitoring system in certain embodiments of the present disclosure. Similar components described in conjunction with FIGS. 2 and 3 are similarly labeled, and description of similar components in conjunction with FIG. 2 above is applicable to the configuration provided in FIG. 3. Referring to FIG. 3, there is provided a voltage regulator 310 operatively coupled to the voltage generator 230. Voltage regulator 310 in certain embodiments is configured to receive the boosted voltage Vchp from the charge pump 210 and provides output voltage Vhv which, as shown in FIG. 3, are provided to the amplifiers including transimpedance amplifier 215, the servo amplifier 235 as well as the two unity gain amplifiers 240, 245. In certain embodiments, the output voltage Vhv of the voltage regulator 310 is 2.8 volts.

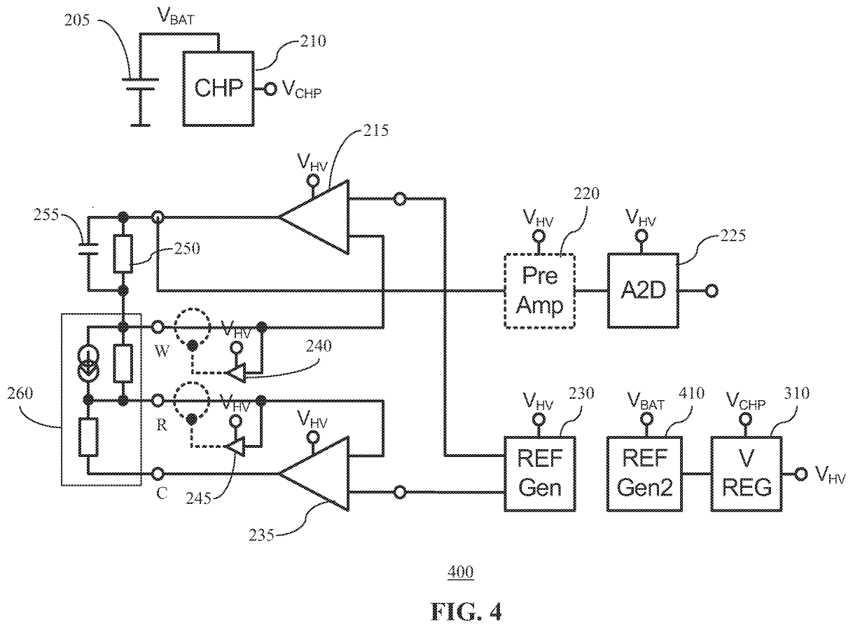

FIG. 4 is yet another illustration of the analog front end architecture for electrical coupling to analyte sensor electrodes in an analyte monitoring system in certain embodiments of the present disclosure. Similar components described in conjunction with FIGS. 2, 3 and 4 are similarly labeled, and description of similar components in conjunction with FIGS. 2 and 3 above are applicable to the configuration provided in FIG. 4. Referring to FIG. 4, compared to the configuration shown in FIG. 3, there is also provided a second reference generator 410 that receives the battery voltage Vbat from battery 205 and is operatively coupled to the voltage regulator 310 as described in conjunction with FIG. 3.

In the manner described above, the configuration shown in FIG. 2 provides for direct voltage measurement from the output of the transimpedance amplifier 215 to the optional pre-amplifier 220, and thereafter processed by the analog to digital converter 225. The configuration shown in FIG. 3 provides for direct voltage measurement using a high voltage regulator as input to the transimpedance amplifier 215 and the servo amplifier 235, as well as the analog to digital converter 225 and preamplifier 220. The voltage regulator 310 receives the charge pump voltage (Vchp) and outputs the regulated voltage (Vhv). However, the regulated voltage (Vhv) output from the voltage regulator 310 shown in FIG. 3 is not provided as input to the reference generator 230, but rather, uses the charge pump voltage (Vchp) which is the boosted voltage from the battery 205. Referring to FIG. 4, the configuration of the analog front end circuitry 400 includes a separate reference generator 410 for the voltage regulator 310 that is driven from the battery 205 voltage (Vbat).

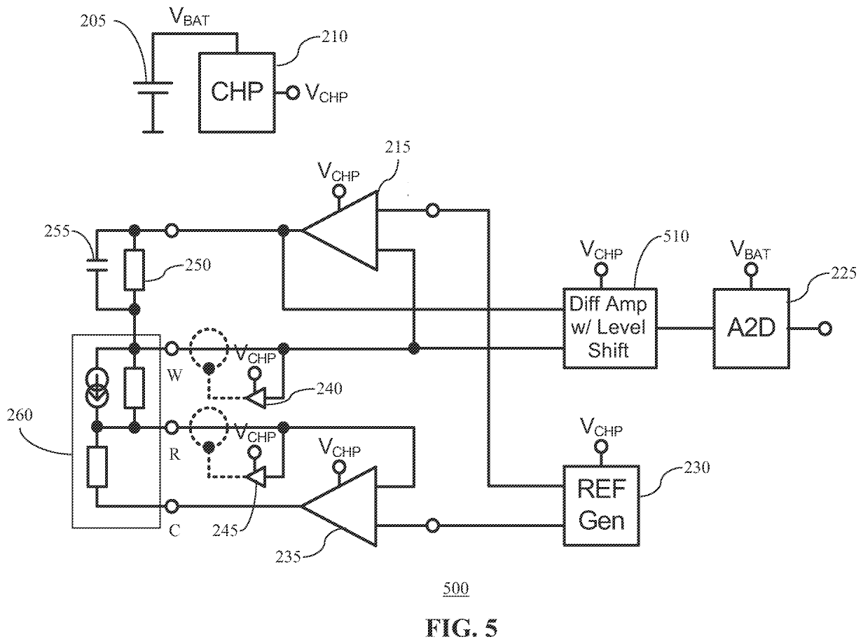

FIG. 5 is yet still another illustration of the analog front end architecture for electrical coupling to analyte sensor electrodes in an analyte monitoring system in certain embodiments of the present disclosure. Similar components described in conjunction with FIGS. 2 and 5 are similarly labeled, and description of similar components in conjunction with FIG. 2 above are applicable to the configuration provided in FIG. 5. Referring to FIG. 5, there is provided a differential amplifier with level shift 510, which is operatively coupled between the output of the transimpedance amplifier 215 and working electrode W of the analyte sensor 260, and the input to the analog to digital converter 225 as shown in FIG. 5, and is configured to provide a differential voltage measurement with a voltage shift down.

More specifically, referring to FIG. 5, in certain embodiments, the voltage at the working electrode W of the analyte sensor 260 is input to the differential amplifier 510 and the output voltage of the transimpedance amplifier 215, and taking the different between the two input voltages, and thereafter, level shifting down the sensor voltage signal from the 2 Volt common mode voltage (which is the output voltage of the transimpedance amplifier 215) to a voltage that is below 1.5 Volts. This allows the analyte sensor 260 to be driven with the battery 205 voltage (Vbat) rather than the voltage from the charge pump 210 (Vchp) resulting in a reduction in noise and power consumption, as the analog to digital converter 225 can be operated from the battery 205 voltage (Vbat) as opposed to the voltage (Vchp) from the charge pump 210.

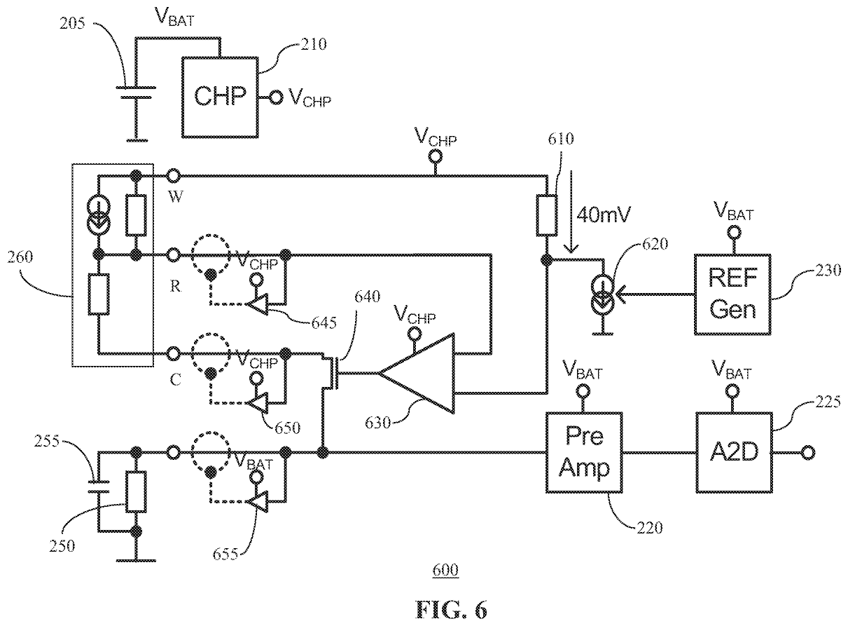

FIG. 6 is yet still a further illustration of the analog front end architecture for electrical coupling to analyte sensor electrodes in an analyte monitoring system in certain embodiments of the present disclosure. Similar components described in conjunction with FIGS. 2 and 6 are similarly labeled, and description of similar components in conjunction with FIG. 2 above are applicable to the configuration provided in FIG. 6. Referring to FIG. 6, a current source 620 is operatively coupled to the reference generator 230 operating at the voltage Vbat from the battery 205, and with resistor 610 operatively coupled to the working electrode W of analyte sensor 260, the poise voltage of 40 mVolts is provided to bias the analyte sensor 260.

As shown in FIG. 6, the resistor 610 is coupled between the working electrode W of the analyte sensor 260 and the input to the amplifier 630, which, in conjunction with the field effect transistor (FET) 640 effectively operates as a current sink. More specifically, the output of the amplifier 630 is coupled to the gate terminal of the FET 640, while the drain terminal of the FET 640 is coupled to the counter electrode C of the analyte sensor 260, and the source terminal of the FET 640 is coupled to the pre-amplifier 220. In this manner, in certain embodiments, the signal path from the analyte sensor 260 to the on body electronics 110 (FIG. 1) is sourced by the battery voltage (Vbat). Furthermore, the configuration shown in FIG. 6 provides for less current consumption without the need for high accuracy components for sensor biasing. Additionally, as shown in FIG. 6, guard trace represented by dotted circles provide leakage production around the electrical connections on the analog front end circuitry 600 of the on body electronics 110 (FIG. 1).

More specifically, referring to FIG. 6, as shown, the battery 205 voltage (Vbat) drives the reference generator 230 to provide the voltage needed at the current source 620 to generate the poise voltage of 40 mVolts for biasing the sensor. As the current flows through the resistor 610, the poise voltage is generated. As discussed above, the FET 640 and the amplifier 630 provide a current sink for the analog front end circuit 600, which, using the charge pump voltage (Vchp) at, for example, 3 Volts, forces the reference electrode R to be maintained at 40 mVolts below the working electrode voltage (maintained at the charge pump voltage Vchp—e.g., 3 Volts). This is achieved by driving the gate terminal of the FET 640 with the output of the amplifier 630. The sensor current, then flows through the FET 640 to the resistor 250 which is input to the pre-amplifier 220 and then to the analog to digital converter 225.

In the manner described in conjunction with FIG. 6, in certain embodiments, all the components, including critical components used for sensor signal measurements, are operated with battery voltage (Vbat). By operating with the battery voltage (Vbat), the configuration shown in FIG. 6 provides for reduced noise and power consumption compared to a configuration that operates from the charge pump voltage (Vchp). Furthermore, in the configuration shown in FIG. 6, the analog front end circuit and the analog to digital converter 225 are referenced to the same reference potential (i.e., ground potential), which provides for simpler calibration of the circuit components and analog to digital converter operation. Further, the configuration shown in FIG. 6 allows for the charge pump noise to be effectively filtered because the impedance from the counter electrode C to the working electrode W is very high, so that a capacitor 255 on the counter electrode C to ground very effectively filters out the noise.

Referring still to FIG. 6, similar to the amplifiers 240, 245 shown conjunction with FIGS. 2-5, amplifiers 645, 650, 655 shown in FIG. 6 each respectively provide guard trace around the analog front end circuit connections by maintaining zero voltage difference between the electrode connections (represented by the broken circular rings). More specifically, amplifiers 645 and 650 are configured to receive the boosted voltage (Vchp) from the charge pump 210, while amplifier 655 is configured to receive the voltage Vbat from the battery 205, and in certain embodiments, each amplifier 645, 650, 655 is configured as a unity gain amplifier or a voltage follower with very high input impedance (e.g., 100 GigaOhms).

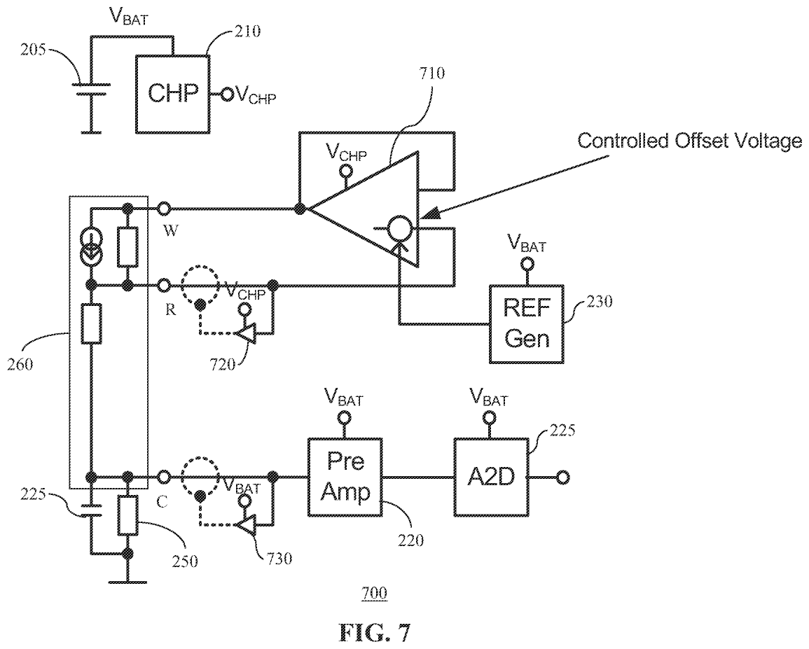

FIG. 7 is yet still another illustration of the analog front end architecture 700 for electrical coupling to analyte sensor electrodes in an analyte monitoring system in certain embodiments of the present disclosure. Similar components described in conjunction with FIGS. 2 and 7 are similarly labeled, and description of similar components in conjunction with FIG. 2 above is applicable to the configuration provided in FIG. 7. Referring to FIG. 7, there is provided a unity gain amplifier 710 whose output is connected to the working electrode W of the analyte sensor 260, and its input coupled to the reference generator 230 operating at the voltage Vbat of the battery 205. In certain embodiments, the unity gain amplifier 710 has a 40 mVolts fixed offset voltage providing for the controlled offset voltage for biasing the sensor 260. Also shown in FIG. 7 are guard traces represented by the dotted circles, but as shown, and for example, compared to the configuration shown in FIG. 6, guard trace around the working electrode W is not needed, effectively reducing the number of guard traces to two rather than three, around each of the three electrodes of the analyte sensor 260. More specifically, as can be seen from FIG. 7, in certain embodiments, amplifiers 720, 730 with very high input impedance (e.g., 100 GigaOhms) provide guard trace around the analog front end circuit connections. Amplifier 720 receives the boosted voltage Vchp from the charge pump 210, while amplifier 730 receives the voltage Vbat from the battery 205, and each amplifier 720, 730 is configured, in certain embodiments as a unity gain amplifier or a voltage follower that is very accurate, such that they maintain zero voltage difference between the electrode connections (for example, the connections for the reference electrode R and the counter electrode C.

In the manner described, in accordance with certain embodiments of the present disclosure, analog front end circuitry configurations are provided to electrically couple to the electrodes of the analyte sensor, and to process the detected sensor signals for further processing. As described above, in certain embodiments, application specific integrated circuits are designed to incorporate the analog front end circuitry to interface with the analyte sensor and also, for subsequent processing of the signals obtained from the analyte sensor for filtering, storage, and/or communication to remote locations or devices such as display device 120 (FIG. 1) in the analyte monitoring system 100.

Certain embodiments of the present disclosure include an analyte monitoring device comprising an analyte sensor having a plurality of sensor electrodes, the analyte sensor having at least a portion in fluid contact with interstitial fluid under a skin layer, and sensor electronics coupled to the sensor electrodes of the analyte sensor and in signal communication with the analyte sensor, the sensor electronics including analog front end circuitry and programmed, or including programmable logic, to process signals generated by the analyte sensor and received by the analog front end circuitry, the signals generated by the analyte sensor corresponding to a monitored analyte level in the interstitial fluid, wherein the analog front end circuitry of the sensor electronics includes a single offset for calibration of the sensor electronics, and further wherein the analog front end circuitry of the sensor electronics are referenced to a reference potential.

In certain aspects, the analog front end circuitry may be provided with a fixed voltage between a working electrode and a reference electrode of the sensor to reference the analog front end circuitry of the sensor electronics to the reference potential.

In certain aspects, the fixed voltage may include the poise voltage associated with the analyte sensor.

In certain aspects, the fixed voltage may include 40 mV.

In certain aspects, the analyte sensor and the sensor electronics may be included within an integrated housing.

In certain aspects, the integrated housing may be configured to be worn on a skin surface of a patient.

In certain aspects, the analyte sensor may be configured to operate for a period of at least 7 days.

In certain aspects, the analyte sensor may be configured to operate for a period of at least 14 days.

In certain aspects, the sensor electronics may include a data communication component to communicate the processed signals to a remote device.

In certain aspects, the data communication component may include a radio frequency (RF) data communication component.

Certain embodiments include an antenna coupled to the data communication component.

In certain aspects, the antenna may include a loop antenna.

Certain embodiments include a guard trace surrounding the plurality of sensor electrodes of the analyte sensor.

Certain embodiments include an analog-to-digital converter, wherein the analog-to-digital converter is referenced to the same reference potential as the analog front end circuitry of the sensor electronics.

Certain embodiments of the present disclosure include a method comprising positioning at least a portion of an analyte sensor in fluid contact with interstitial fluid under a skin layer, the analyte sensor having a plurality of sensor electrodes, coupling sensor electronics to the sensor electrodes of the analyte sensor, wherein the sensor electronics are in signal communication with the analyte sensor, the sensor electronics including analog front end circuitry, and processing, using the sensor electronics, signals generated by the analyte sensor and received by the analog front end circuitry, the signals generated by the analyte sensor corresponding to a monitored analyte level in the interstitial fluid, wherein the analog front end circuitry of the sensor electronics includes a single offset for calibration of the sensor electronics, and further wherein the analog front end circuitry of the sensor electronics are referenced to a reference potential.

In certain aspects, the analog front end circuitry may be provided with a fixed voltage between a working electrode and a reference electrode of the sensor to reference the analog front end circuitry of the sensor electronics to the reference potential.

In certain aspects, the fixed voltage may include the poise voltage associated with the analyte sensor.

In certain aspects, the fixed voltage may include 40 mV.

In certain aspects, the analyte sensor and the sensor electronics may be included within an integrated housing.

In certain aspects, the integrated housing may be configured to be worn on a skin surface of a patient.

In certain aspects, the analyte sensor may be configured to operate for a period of at least 7 days.

In certain aspects, the analyte sensor may be configured to operate for a period of at least 14 days.

In certain aspects, the sensor electronics may include a data communication component to communicate the processed signals to a remote device.

In certain aspects, the data communication component may include a radio frequency (RF) data communication component.

Certain embodiments include coupling an antenna to the data communication component.

In certain aspects, the antenna may include a loop antenna.

Certain embodiments include surrounding the plurality of sensor electrodes of the analyte sensor with a guard trace.

Certain embodiments include operatively coupling an analog-to-digital converter to the analyte front end circuitry of the sensor electronics, wherein the analog-to-digital converter is referenced to the same reference potential as the analog front end circuitry.

Various other modifications and alterations in the structure and method of operation of the embodiments of the present disclosure will be apparent to those skilled in the art without departing from the scope and spirit of the present disclosure. Although the present disclosure has been described in connection with certain embodiments, it should be understood that the present disclosure as claimed should not be unduly limited to such embodiments. It is intended that the following claims define the scope of the present disclosure and that structures and methods within the scope of these claims and their equivalents be covered thereby.

1-20. (canceled)

21. An analyte monitoring device, comprising:
an analyte sensor having a plurality of sensor electrodes including a working electrode, a reference electrode, and a counter electrode, the analyte sensor having at least a portion configured to be in contact with fluid under a skin surface, wherein the plurality of sensor electrodes generate at least one signal corresponding to an analyte level in the fluid;
a first amplifier comprising a first input, a second input and an output, wherein the amplifier is configured such that the first input receives a signal from the working electrode, the second input receives a first non-zero reference voltage, and the output produces an output voltage;
an analog-to-digital converter configured to convert a signal representative of the output voltage to digital form;
a second amplifier comprising a first input, a second input, and an output, wherein the amplifier is configured such that the first input receives a signal from the reference electrode, the second input receives a second non-zero reference voltage, and the output is electrically coupled with the counter electrode, wherein the first non-zero reference voltage is greater than the second non-zero reference voltage; and
a guard trace for the working electrode.

22. The analyte monitoring device of claim 21, wherein both the first amplifier and the second amplifier receive a supply voltage, wherein the first non-zero reference voltage and the second non-zero reference voltage are each less than the supply voltage.

23. The analyte monitoring device of claim 22, further comprising a battery and a charge pump, wherein the charge pump is configured to generate the supply voltage from a voltage of the battery.

24. The analyte monitoring device of claim 21, further comprising a feedback path having a capacitor, the feedback path connected between the first input and the output of the first amplifier.

25. The analyte monitoring device of claim 21, further comprising a reference generator configured to generate the first non-zero reference voltage and the second non-zero reference voltage.

26. The analyte monitoring device of claim 21, wherein a difference between the first non-zero reference voltage and the second non-zero reference voltage is fixed.

27. The analyte monitoring device of claim 26, wherein the difference includes 40 millivolts.

28. The analyte monitoring device of claim 21, further comprising an on body housing comprising an interior that holds a portion of the analyte sensor, and an adhesive layer configured to adhere to the skin surface.

29. An analyte monitoring device, comprising:
an analyte sensor having a plurality of sensor electrodes including a working electrode, a reference electrode, and a counter electrode, the analyte sensor having at least a portion configured to be in contact with fluid under a skin surface, wherein the plurality of sensor electrodes generate at least one signal corresponding to an analyte level in the fluid;
a first amplifier comprising a first input, a second input and an output, wherein the amplifier is configured such that the first input receives a signal from the working electrode, the second input receives a first non-zero reference voltage, and the output produces an output voltage;
an analog-to-digital converter configured to convert a signal representative of the output voltage to digital form;
a second amplifier comprising a first input, a second input, and an output, wherein the amplifier is configured such that the first input receives a signal from the reference electrode, the second input receives a second non-zero reference voltage, and the output is electrically coupled with the counter electrode; and
a guard trace for the working electrode.

30. The analyte monitoring device of claim 29, wherein both the first amplifier and the second amplifier receive a supply voltage, wherein the first non-zero reference voltage and the second non-zero reference voltage are each less than the supply voltage.

31. The analyte monitoring device of claim 30, further comprising a battery and a charge pump, wherein the charge pump is configured to generate the supply voltage from a voltage of the battery.

32. The analyte monitoring device of claim 29, wherein the first non-zero reference voltage is greater than the second non-zero reference voltage.

33. The analyte monitoring device of claim 29, feedback path having a capacitor, the feedback path connected between the first input and the output of the first amplifier

34. The analyte monitoring device of claim 29, further comprising a reference generator configured to generate the first non-zero reference voltage and the second non-zero reference voltage.

35. The analyte monitoring device of claim 29, wherein a difference between the first non-zero reference voltage and the second non-zero reference voltage is fixed.

36. The analyte monitoring device of claim 35, wherein the difference includes 40 millivolts.

37. The analyte monitoring device of claim 29, further comprising a pre-amplifier configured to buffer the output voltage from the first amplifier and provide the buffered output voltage to the analog-to-digital converter.

38. An analyte monitoring system, comprising:
an analyte monitoring device, comprising:
an analyte sensor having a plurality of sensor electrodes including a working electrode, a reference electrode, and a counter electrode, the analyte sensor having at least a portion configured to be in contact with fluid under a skin surface, wherein the plurality of sensor electrodes generate at least one signal corresponding to an analyte level in the fluid;
sensor electronics comprising:
a first amplifier comprising a first input, a second input and an output, wherein the amplifier is configured such that the first input receives a signal from the working electrode, the second input receives a first non-zero reference voltage, and the output produces an output voltage;
an analog-to-digital converter configured to convert a signal representative of the output voltage to digital form;
a second amplifier comprising a first input, a second input, and an output, wherein the amplifier is configured such that the first input receives a signal from the reference electrode, the second input receives a second non-zero reference voltage, and the output is electrically coupled with the counter electrode; and
a guard trace for the working electrode; and
a housing including the analyte sensor, the sensor electronics, and an adhesive layer configured to adhere to the skin surface; and
an insertion device configured to transcutaneously position a portion of the analyte sensor through the skin surface and in contact with the fluid, and to position the housing and adhesive layer of the analyte monitoring device on the skin surface.

39. The analyte monitoring system of claim 38, wherein the analyte monitoring device is configured to communicate with a smart phone.

40. The analyte monitoring system of claim 39, wherein the pre-amplifier has an impedance that compensates for signal variation in the output of the first amplifier.
